(19) United States
(12) Patent Application Publication
(10) Pub. No.: US 2003/0232978 A1
(43) Pub. Date: Dec. 18, 2003

(54) Reagents that facilitate the purification of compounds synthesized on a solid support

(76) Inventors: Peter H. Seeberger, Cambridge, MA (US); Michael C. Hewitt, Cambridge, MA (US); Emma R. Palmacci, Cambridge, MA (US); Obdiah J. Plante, Danvers, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/219,129

(22) Filed: Aug. 15, 2002

Related U.S. Application Data
(60) Provisional application No. 60/314,909, filed on Aug. 24, 2001.

(51) Int. Cl. C08B 37/00; C07H 21/04

(52) U.S. Cl. 536/25.3; 536/53; 525/54.2

(57) ABSTRACT

Disclosed is a novel capping-and-tagging (“cap-tag”) strategy that aids in the purification of compounds assembled by multi-step synthetic methods. The incorporation of a unique cap-tag into undesired intermediates during a multi-step synthesis allows for the straightforward removal of these undesired compounds at the conclusion of the synthetic protocol by several methods based on affinity chromatography and scavenging resins. Importantly, this invention may be exploited in any automated synthetic protocol. In preferred embodiments, the cap-tag system is exploited for the purification of carbohydrates prepared via automated solid-phase methods.

### RELATED APPLICATIONS

This application claims the benefit of priority to U.S. Provisional Patent Application serial No. 60/314,909, filed Aug. 24, 2001.

### BACKGROUND OF THE INVENTION

Typically, the purification of synthetic compounds is a difficult and time-consuming process. The advent of automated solid-phase synthesis of biopolymers and the like has greatly simplified the assembly process by allowing for multi-step syntheses to be performed without the need for the purification of synthetic intermediates. However, the buildup of myriad unwanted by-products during an automated solid-phase synthesis often renders even more challenging the final purification step.

Specifically, in the context of biopolymer synthesis, the removal from the desired product of sequences different by just one unit (so-called “n−1 products”), which stem from incomplete conversion at any stage of the synthesis, can be very difficult. Notably, a number of methods have been reported to isolate and purify biopolymers, including biopolymers produced by chemical synthesis or recombinant DNA techniques. For example, known purification methods include centrifugation, column chromatography and electrophoresis. While these methods can produce a purified biopolymer, they each require one or more additional and often burdensome purification steps after initial purification of the biopolymer. Moreover, in many current isolation and purification procedures, a significant amount of the crude biopolymer is lost during the procedure resulting in reduced yields. Further, because automated syntheses offer many advantages over traditional step-wise syntheses, the purification of compounds resulting from automated multi-step syntheses is an area of great importance in both academic and industrial environments. The following discussion of these issues in the context of polynucleotides is illustrative.

Automated Polynucleotide Synthesis

Many advances in molecular biology have been facilitated by the development of reliable and convenient methods for synthesizing polynucleotides, e.g. Itakura, Science, Vol. 209, pgs. 1401-1405 (1980); and Wallace et al, pgs. 631-663, in Scouten, ed. Solid Phase Biochemistry (John Wiley & Sons, New York, 1982). As the use of synthetic polynucleotides has increased, the demand for even greater convenience in the preparation of pure, ready-to-use polynucleotides has also increased. This demand has stimulated the development of many improvements in the basic procedures for solid phase synthesis, e.g. Sinha et al, Nucleic Acids Research, Vol. 12, pgs. 4539-4557 (1984) (beta-cyanoethyl in phosphoramidite chemistries); Froehler et al, Tetrahedron Letters, Vol. 27, pgs. 469-472 (1986) (H-phosphonate chemistry); Germann et al, Anal. Biochem., Vol. 165, pgs. 399-405 (1987); and Ikuta et al, Anal. Chem., Vol. 56, pgs. 2253-2256 (1984) (rapid purification of synthetic oligonucleotides by way of trityl moieties); Molko et al, European patent publication 241363 dated Apr. 3, 1987 (improved base-labile acyl protection groups for exocyclic amines), and the like.

In spite of such progress, difficulties are still encountered in current methods of polynucleotide synthesis and purification. For example, H-phosphonate and phosphoramidite monomers readily degrade in the presence of even trace amounts of water. The presence of water-degraded reactants leads to less pure final products and to more expensive syntheses. Further, poly(nucleic acid)s (PNAs) are now routinely synthesized from monomers (PNA synthons) protected according to the t-Boc/benzyl protection strategy, wherein the backbone amino group of the growing polymer is protected with the t-butyloxycarbonyl (t-Boc) group and the exocyclic amino groups of the nucleobases, if present, are protected with the benzyloxycarbonyl (benzyl) group. When acid is used to completely remove the more acid labile t-Boc protecting groups, there is a potential that a percentage of benzyl groups will also be removed contemporaneously.

Specifically, the t-Boc protecting group must be removed from the amino group backbone during each synthetic cycle so the next monomer can be attached to the backbone at the free amino site thereby allowing the polymeric chain to grow. The deprotection of the t-Boc amino protected backbone is accomplished using a strong acid such as trifluoroacetic acid. During this deprotection and subsequent construction of the PNA oligomer, removal of the nucleobase side chain protecting groups, i.e., the benzyls, is undesirable. However, trifluoroacetic acid is potentially strong enough to deprotect prematurely a percentage of the side chain benzyl groups, thereby introducing the possibility of polymer branching and reducing the overall yield of desired product. Importantly, the purification of the final product is made more difficult by the presence of branched impurities.

### SUMMARY OF THE INVENTION

Disclosed is a novel capping-and-tagging (“cap-tag”) strategy that aids in the purification of compounds assembled by multi-step synthetic methods. The incorporation of a unique cap-tag into undesired intermediates during a multi-step synthesis allows for the straightforward removal of these undesired compounds at the conclusion of the synthetic protocol by several methods based on affinity chromatography and scavenging resins. Importantly, this invention may be exploited in any automated synthetic protocol. In preferred embodiments, the cap-tag system is exploited for the purification of carbohydrates prepared via automated solid-phase methods.

### BRIEF DESCRIPTION OF THE FIGURES

FIG. 1 depicts schematically a solid-phase synthetic protocol, including illustrations of a way in which (n−1) sequences are introduced into the product mixture, and the use of a method of the present invention to facilitate isolation and purification of the desired product.

FIG. 2 depicts generalized biopolymers functionalized with each of two embodiments of the compounds of the present invention.

FIG. 3 depicts schematically the attachment to a carbohydrate of each of two embodiments of the compounds of the present invention, and subsequent purification procedures exploiting the attached compounds of the present invention.

FIG. 4 depicts a reiterative solid-phase synthetic protocol that includes steps incorporating a compound of the present invention and using that compound as the basis for removing from the product impurities and by-products.

FIG. 5 depicts two embodiments of the compounds of the present invention.

### DETAILED DESCRIPTION OF THE INVENTION

The strategy disclosed herein uses unique cap-tag procedures that address a major challenge in the synthesis of biopolymers, e.g., oligosaccharides; namely, the challenge of purification. Currently used purification methods rely on traditional techniques such as HPLC or capillary electrophoresis for the isolation and separation of product biopolymers, e.g., carbohydrates. The cap-tag method allows for the majority of unwanted biopolymer sequences to be separated from the desired product without the need for tedious purification steps. This ease of use enables the cap-tag method to be performed on a much larger scale that other purification methods.

Disclosed is a novel capping-and-tagging (“cap-tag”) strategy that aids in the purification of compounds assembled by multi-step methods. The incorporation during the synthesis of a unique cap-tag on undesired intermediates allows for the starightforward removal of these sequences at the conclusion of the synthetic protocol by several methods based on affinity chromatography and scavenging resins. Importantly, this invention may be exploited in any automated synthetic protocol. In preferred embodiments, the cap-tag system is exploited for the purification of carbohydrates prepared via automated solid-phase methods.

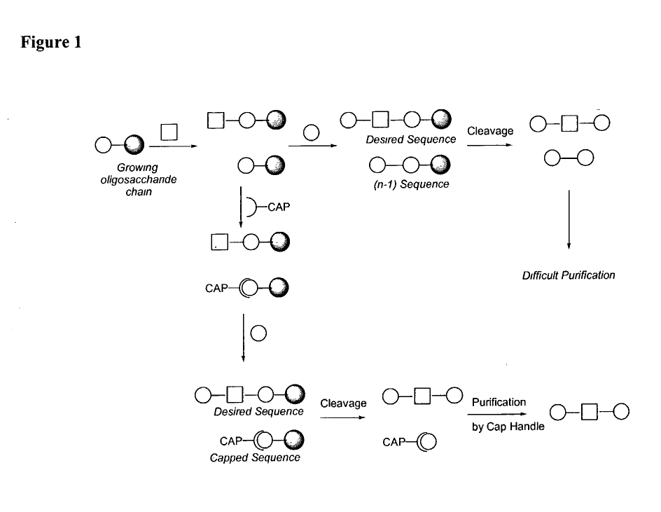

Exemplary embodiments of the present invention are provided by the following two novel cap-tag compounds and methods that aid in the purification of oligosaccharides assembled by automated solid-phase synthesis. Following each coupling event unreacted hydroxyl groups that may give rise to deletion sequences are subjected to a capping reagent that renders these sites silent in subsequent couplings. See FIG. 1. The cap-tags also function as a handle to readily separate all unwanted capped and tagged sequences from the desired untagged products. The introduction of these cap-tags into the automated solid-phase synthesis of biopolymers, e.g., oligosaccharides, greatly simplifies post-synthetic work-up and purification of oligosaccharides.

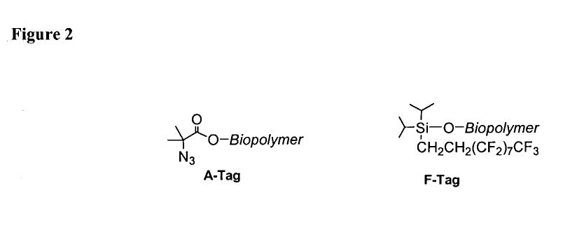

Two preferred embodiments of cap-tags are described in detail below. First, an α-azido isobutyric ester cap (A-Tag) that can be removed with an isocyanate functionalized scavenger resin after reduction to the corresponding amine. See FIG. 2. Second, a fluorous silyl ether cap (F-Tag) that allows for the removal of tagged sequences by filtration through fluorous silica gel. See FIG. 2.

Cap-Tag Design

A number of factors contribute to the success of a particular cap-tag. First, the capping reagents must be highly reactive with unreacted positions on the growing biopolymer, e.g., they must react rapidly with hydroxyl groups that were not glycosylated in the previous step of an automated oligosaccharide synthesis. Second, the linkage formed between the unreacted position, e.g., hydroxyl group of an oligosaccharide, of the growing biopolymer and the cap-tag must be stable to subsequent coupling cycles. Third, any cap-tag utilized must be “orthogonal” to the protecting group scheme used in the biopolymer synthesis; in other words, the cap-tag must be unreactive under the conditions required to protect and deprotect positions in the growing biopolymer during the automated synthesis. Fourth, the cap-tag must also comprise a functional group or groups that allow for facile separation, using standard, preferably large-scale, purification techniques, of the capped-tagged “n−1 products” from the full-length products; that is, the cap-tag must contain a functional group not present in the full-length product, which functional group interacts, e.g., strongly, with a standard purification medium, enabling straightforward removal of the capped-tagged “n−1 products” from the product mixture.

Installation and Separation of Cap-Tags

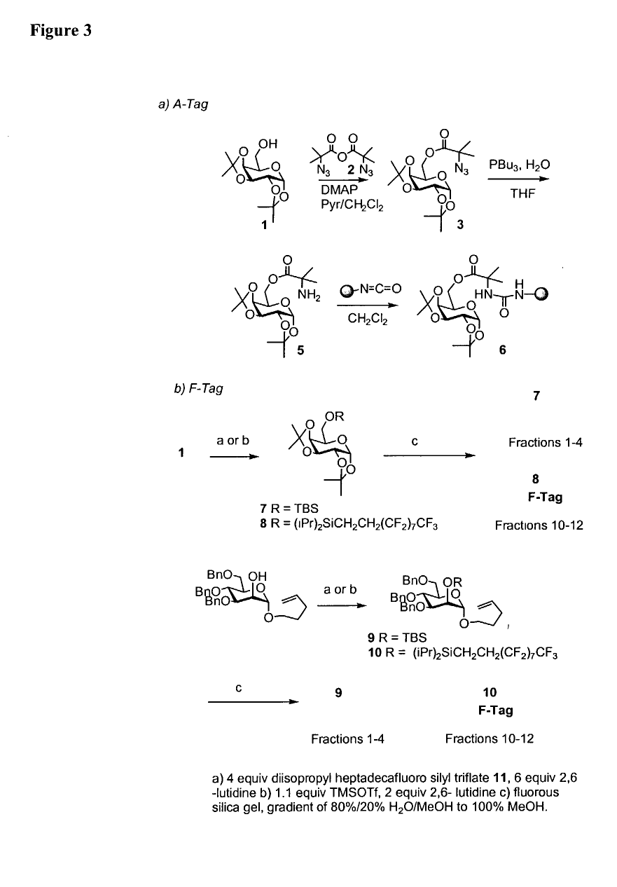

For example, the A-Tag is readily installed on hydroxyl groups using the A-Tag anhydride 2. See FIG. 3a. The resulting ester linkage is stable to several routine transformations in biopolymer synthesis, e.g., carbohydrate synthesis. Reaction of compounds similar to 3, differentiated with an A-Tag, with a reducing agent affords a primary amine. Selective removal of amines from the reaction mixture may be accomplished by addition of an amine scavenging resin; for example, polymer bound isocyanates or tosylates. Filtration of the scavenging resin affords only untagged compounds, while the A-Tagged compounds remain covalently bound to the resin.

In another embodiment, the F-Tag is easily installed by silylation of hydroxyl groups, e.g., of a growing oligosaccharide, with a densely fluorinated alkyl side-chain. See FIG. 3b. Synthetic intermediates comprising F-Tags are stable to strongly acidic and basic reaction conditions. The removal from the product mixture of intermediates with the F-Tag is accomplished by filtration through a portion of fluorous silica. Untagged compounds elute in 20% aqueous methanol, while the tagged fluorinated compounds remain bound to the fluorous silica gel. Recovery of the F-Tagged compounds can be achieved by elution with methanol.

Use of Cap-Tags in Automated Solid-Phase Oligosaccharide Synthesis

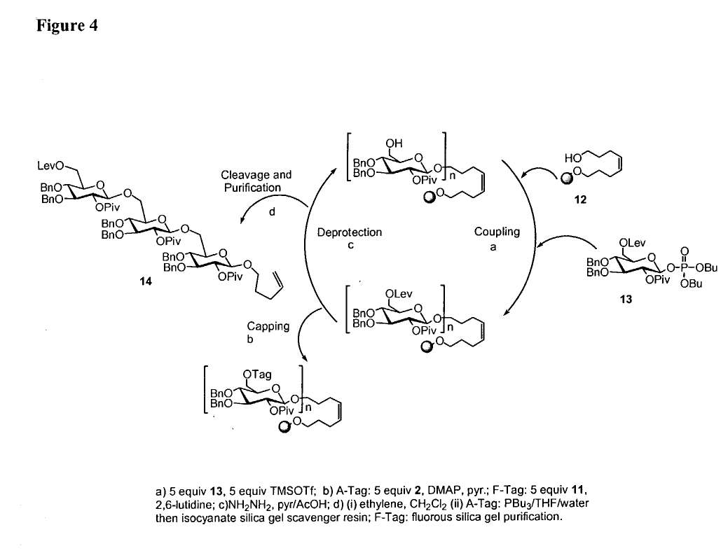

In a preferred embodiment, the incorporation of a cap-tag step into protocols for automated solid-phase carbohydrate synthesis greatly facilitates the purification of the desired sequences. To illustrate, following glycosylation, the remaining unreacted hydroxyl groups are capped with either the A-Tag or F-Tag. See FIG. 4. Next, deprotection of a unique protecting group is accomplished without reaction of the chosen tag. Elongation of the oligosaccharide is then continued by another glycosylation event with subsequent capping of any unreacted hydroxyl groups. Repetition of this sequence of events, i.e., glycosylation, capping-tagging, and deprotection, provides a convenient method for the assembly of carbohydrates. Removal of any unwanted capped-tagged sequences has been easily accomplished using the aforemntioned A-Tag or F-Tag separation procedures.

Definitions

For convenience, certain terms employed in the specification, examples, and appended claims are collected here.

The term “heteroatom” as used herein means an atom of any element other than carbon or hydrogen. Preferred heteroatoms are boron, nitrogen, oxygen, phosphorus, sulfur and selenium.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, J. March, Advanced Organic Chemistry, McGraw Hill Book Company, New York, (1977 edition) pp. 251-259. The Hammett constant values are generally negative for electron donating groups (σ[P]=−0.66 for NH₂) and positive for electron withdrawing groups (σ[P]=0.78 for a nitro group), σ[P] indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formula:

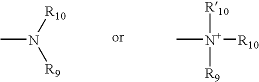

wherein R₉, R′₁₀ and R′₁₀ each independently represent a group permitted by the rules of valence.

The term “acylamino” is art-recognized and refers to a moiety that can be represented by the general formula:

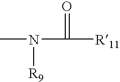

wherein R₉ is as defined above, and R′₁₁ represents a hydrogen, an alkyl, an alkenyl or —(CH₂)m—R₈, where m and R₈ are as defined above.

The term “amido” is art recognized as an amino-substituted carbonyl and includes a moiety that can be represented by the general formula:

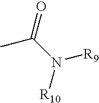

wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)m—R₈, wherein m and R₈ are defined above. Representative alkylthio groups include methylthio, ethyl thio, and the like.

The term “carbonyl” is art recognized and includes such moieties as can be represented by the general formula:

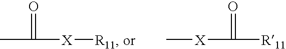

wherein X is a bond or represents an oxygen or a sulfur, and R′₁₁ represents a hydrogen, an alkyl, an alkenyl, —(CH₂)m—R₈ or a pharmaceutically acceptable salt, R′₁₁ represents a hydrogen, an alkyl, an alkenyl or —(CH₂)m—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiolcarbonyl” group. Where X is a sulfur and R₁₁ or R′₁₁ is not hydrogen, the formula represents a “thiolester.” Where X is a sulfur and R₁₁ is hydrogen, the formula represents a “thiolcarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)m—R₈, where m and R₈ are described above.

The term “sulfonate” is art recognized and includes a moiety that can be represented by the general formula:

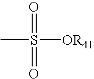

in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The term “sulfate” is art recognized and includes a moiety that can be represented by the general formula:

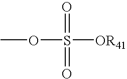

in which R₄₁ is as defined above.

The term “sulfonylamino” is art recognized and includes a moiety that can be represented by the general formula:

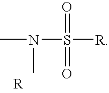

The term “sulfamoyl” is art-recognized and includes a moiety that can be represented by the general formula:

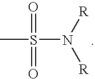

The term “sulfonyl”, as used herein, refers to a moiety that can be represented by the general formula:

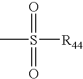

in which R₄₄ is selected from the group consisting of hydrogen, alkyl, alkenyl, alkynyl, cycloalkyl, heterocyclyl, aryl, or heteroaryl.

The term “sulfoxido” as used herein, refers to a moiety that can be represented by the general formula:

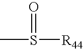

in which R₄₄ is selected from the group consisting of hydrogen, alkyl, alkenyl, alkynyl, cycloalkyl, heterocyclyl, aralkyl, or aryl.

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)m—R₇, m and R₇ being defined above.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof, wherein one or more simple variations of substituents are made which do not adversely affect the efficacy of the compound in binding to monoamine transporters. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

Compounds of the Invention

Generally, a capping-tagging compound of the present invention comprises: a first functional group capable of forming a covalent bond, e.g., a amide linkage, an ester linkage, an alkylamine linkage, or an ether linkage, with a free nucleophilic site on a biopolymer tethered to a solid support; and a second functional group capable of forming a covalent bond or non-covalent interaction with a solid material, e.g., an affinity chromatography material, or a scavenging resin, or said second functional group is capable of being efficiently transformed to a third functional group, which third functional group is capable of forming a covalent bond or non-covalent interaction with a solid material.

This second or third functional group may be referred to as a “recognition moiety.” The recognition moiety can interact with the aforementioned solid material via either attractive or repulsive mechanisms. In one exemplary embodiment, the aforementioned solid material and the recognition moiety form an intimately associated pair by, for example, covalent bonding, ionic bonding, ion pairing, van der Waals association and the like. In another exemplary embodiment, the aforementioned solid material and recognition moiety interact by a repulsive mechanism such as incompatible steric characteristics, charge-charge repulsion, hydrophilic-hydrophobic interactions and the like. In the latter embodiment, the presence of the cap-tag on the “n—1 products” causes them to pass more quickly through the solid material than the desired product.

In certain embodiments, the recognition moiety is a chelating agent, crown ether or cyclodextrin; in such embodiments, host-guest chemistry will dominate the interaction between the recognition moiety and the aforementioned solid material. The use of host-guest chemistry allows a great degree of recognition-moiety-solid-material specificity to be realized.

In certain embodiments, a capping-tagging compound of the present invention comprises a carboxylic acid moiety; and an azide moiety. Using chemistry known to one of ordinary skill in the art of organic chemistry, the carboxylic acid moiety of such a capping-tagging compound can be used to form a covalent bond, i.e., an amide or ester linkage, with a reactive nucleophilic site, i.e., an amine or an alcohol, of the biopolymer intermediate tethered to the solid support, thereby blocking further elaboration of the biopolymer intermediate at that site. Again using chemistry known to one of ordinary skill in the art of organic chemistry, the azide moiety of such a capping-tagging compound can be reduced upon completion of the synthesis of the desired biopolymer to give an amine, which amine can serve as the basis for separating the capped-tagged n—1 products from the desired products.

In other embodiments, a capping-tagging compound of the present invention comprises a silyl halide moiety; and a fluorinated hydrocarbon moiety. Using chemistry known to one of ordinary skill in the art of organic chemistry, the silyl halide moiety of such a capping-tagging compound can be used to form a covalent bond, i.e., a silyl ether, with a reactive alcohol of the biopolymer intermediate tethered to the solid support, thereby blocking further elaboration of the biopolymer intermediate at that site. Again using chemistry known to one of ordinary skill in the art of organic chemistry, the fluorinated hydrocarbon moiety of such a capping-tagging compound can serve as the basis for separating the capped-tagged n−1 products from the desired products.

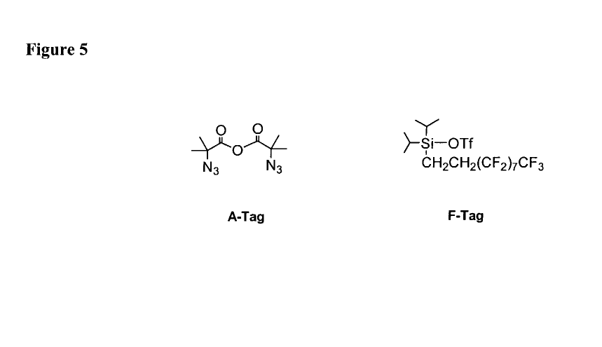

Two preferred embodiments of the capping-tagging compounds are described in detail below. First, an α-azido isobutyric acid anhydride-based cap (A-Tag) that can be removed with an isocyanate functionalized scavenger resin after reduction to the corresponding amine. See FIG. 5. Second, a fluorous silyl triflate-based cap (F-Tag) that allows for the removal of tagged sequences by filtration through fluorous silica gel. See FIG. 5.

Methods of the Invention

In certain embodiments, the present invention relates to a method of facilitating the purification of compounds synthesized on a solid support, comprising the step of: treating a compound covalently tethered to a solid support, wherein said compound comprises a nucleophilic functional group, with a capping-tagging reagent selected from the group consisting of activated azido-carboxylic acids, azido-carboxylic acid anhydrides, fluoroalkylsilyl sulfonates, and fluoroalkylsilyl halides, thereby forming a second compound tethered to a solid support.

In certain embodiments, the compound covalently tethered to a solid support is a biopolymer.

In certain embodiments, the compound covalently tethered to a solid support is an oligosaccharide.

In certain embodiments, the compound covalently tethered to a solid support is an oligonucleotide.

In certain embodiments, the nucleophilic functional group is an alcohol.

In certain embodiments, the nucleophilic functional group is an amine.

In certain embodiments, the capping-tagging reagent is an activated 2-azido-carboxylic acid or 2-azido-carboxylic acid anhydride.

In certain embodiments, the capping-tagging reagent is a 2-azido-carboxylic acid anhydride.

In certain embodiments, the capping-tagging reagent is 2-azido-isobutyric acid anhydride.

In certain embodiments, the capping-tagging reagent is a fluoroalkylsilyl triflate.

In certain embodiments, the capping-tagging reagent is a fluoroalkyldiisopropylsilyl triflate.

In certain embodiments, the capping-tagging reagent is heptadecafluorodecyldiisopropylsilyl triflate.

In certain embodiments, the compound covalently tethered to a solid support is an oligosaccharide; and the nucleophilic functional group is an alcohol.

In certain embodiments, the compound covalently tethered to a solid support is an oligonucleotide; and the nucleophilic functional group is an alcohol.

In certain embodiments, the compound covalently tethered to a solid support is an oligosaccharide; the nucleophilic functional group is an alcohol; and the capping-tagging reagent is 2-azido-isobutyric acid anhydride.

In certain embodiments, the compound covalently tethered to a solid support is an oligonucleotide; the nucleophilic functional group is an alcohol; and the capping-tagging reagent is 2-azido-isobutyric acid anhydride.

In certain embodiments, the compound covalently tethered to a solid support is an oligosaccharide; the nucleophilic functional group is an alcohol; and the capping-tagging reagent is heptadecafluorodecyldiisopropylsilyl triflate.

In certain embodiments, the compound covalently tethered to a solid support is an oligonucleotide; the nucleophilic functional group is an alcohol; and the capping-tagging reagent is heptadecafluorodecyldiisopropylsilyl triflate.

In certain embodiments, the present invention relates to any of the aformentioned methods of facilitating the purification of compounds synthesized on a solid support, further comprising the steps of: cleaving said tether of said second compound covalently tethered to a solid support to produce a compound in solution; and purifying said compound in solution using affinity chromatography or a scavenging resin.

In certain embodiments, the present invention relates to any of the aformentioned methods, wherein said compound in solution is purified using affinity chromatography with a fluorous silica gel as a stationary phase.

In certain embodiments, the present invention relates to any of the aformentioned methods, wherein said compound in solution is purified using a scavenging resin comprising an isocyanate, isothiocyanate, alkyl halide, aryl halide, aryl boronic acid, alkyl sulfonate, aldehyde, carboxylic acid halide, carboxylic acid anhydride, or sulfonyl halide.

In certain embodiments, the present invention relates to any of the aformentioned methods, wherein said compound in solution is purified using a scavenging resin comprising an isocyanate or isothiocyanate.

Affinity Chromatography

Affinity chromatography enables the efficient isolation of species such as biological molecules or biopolymers by utilizing their recognition sites for certain supported chemical structures with a high degree of selectivity. Affinity chromatographic methods have utilized materials of varying chemical structure as supports. For example, agarose gels and cross-linked agarose gels have been the most widely used support materials. Their hydrophilicity makes them relatively free of nonspecific binding, but their compressibility makes them less attractive as carriers in large scale processing, such as in manufacturing. Controlled-pore glass (CPG) beads have also been used in affinity chromatography. Although high throughputs can be obtained with columns packed with CPG, this carrier is even more expensive than agarose beads. Cellulose particles have also been used by immunochemists for synthetic affinity sorbents. However, compared to agarose gels, cellulose particles are formed with more difficulty and therefore, have received less attention in the preparation of affinity sorbents for enzymes. Cellulose, however, is perhaps the least expensive of all support matrices. Two lesser-used support matrices are polyacrylamide gel beads and Sephadex.TM. gel beads made from dextran and epichlorohydrin. Although convenient methods have been developed for using them, the softness of these beads yields poor column packings, and their low molecular porosity yields a sorbent with poor ligand availability to the ligate.

Scavenging Resins

Purification of reaction mixtures after organic synthesis is often performed using flash chromatography, which is a chromatographic technique used to separate or fractionate products of interest using pre-packed silica columns. However, the purification achieved by flash chromatography can be adversely affected by the presence of excess reagents in the reaction mixture. Removal of excess reagents prior to flash chromatography can improve the efficiency of the separation and the purification achieved. One approach to removing impurities from a product mixture, e.g., prior to chromatography, is the use of scavenger resins.

The use of polymer-bound reagents to assist in the purification step of reaction mixtures and products has been gaining momentum over the past few years. Scavenger resins are added to the pot after the reaction is completed in order to quench and selectively bind to excess reagents or byproducts. Filtration of the reaction mixture separates the polymer-bound impurities from the product yielding pure compounds. Scavenger resins ease the purification process by providing an alternative or supplement to extractions and chromatography. Multiple scavenger resins can be used in parallel; thus, multiple reagents and byproducts and be removed concurrently. The choice of scavenger resin is determined by the functionality of the impurity. For example, to remove a by-product with a nucleophilic functional group, a scavenger resin with an electrophilic functional group would be used.

Polystyrene is used as the polymeric backbone for some scavenger resins. Lightly crosslinked polystyrenes scavenger resins typically require the use of solvents that will swell the resin to allow reagents to access the resin-bound functional groups. In cases where the solvent does not adequately swell the resin, it may be necessary to add a co-solvent that is compatible with the resin, e.g. THF. Highly crosslinked macroporous resins swell significantly less in solvent and they are not dependent on swelling to be effective, unlike their lightly crosslinked polystyrene-bound counterparts. Instead, reagents diffuse through the pore structure to reach reactive sites. For this reason, highly crosslinked polystyrene resins can be used in confined volumes where swelling would be a problem and can be used with a wider variety of solvents.

In brief, scavenger resins contain a chemical group that will react only with the by-products or excess reagent that the chemist wants to remove from the reaction or product mixture. The by-product or reagent sticks to the resin, but the product stays in the solution. By filtering the resin, the chemist separates the desired product from the undesired by-product.

Capture resins are similar to scavenger resins, except capture resins selectively and reversibly bind the desired product instead of undesired compounds. After a reaction has finished, the chemist adds a capture resin that binds the product. By filtering and washing the resin, the chemist removes all of the by-products, excess reagents, and soluble impurities. When the product is released from the resin, it will contain only minor amounts of impurities.

Combinatorial Libraries

The subject compounds and methods readily lend themselves to the creation of combinatorial libraries of compounds for the screening of pharmaceutical, agrochemical or other biological or medically-related activity or material-related qualities. A combinatorial library for the purposes of the present invention is a mixture of chemically related compounds which may be screened together for a desired property; said libraries may be in solution or covalently linked to a solid support. The preparation of many related compounds in a single reaction greatly reduces and simplifies the number of screening processes which need to be carried out. Screening for the appropriate biological, pharmaceutical, agrochemical or physical property may be done by conventional methods.

Diversity in a library can be created at a variety of different levels. For instance, the substrate aryl groups used in a combinatorial approach can be diverse in terms of the core aryl moiety, e.g., a variegation in terms of the ring structure, and/or can be varied with respect to the other substituents.

A variety of techniques are available in the art for generating combinatorial libraries of small organic molecules. See, for example, Blondelle et al. (1995) Trends Anal. Chem. 14:83; the Affymax U.S. Pat. Nos. 5,359,115 and 5,362,899: the Ellman U.S. Pat. No. 5,288,514: the Still et al. PCT publication WO 94/08051; Chen et al. (1994) JACS 116:2661: Kerr et al. (1993) JACS 115:252; PCT publications WO92/10092, WO93/09668 and WO91/07087; and the Lemer et al. PCT publication WO93/20242). Accordingly, a variety of libraries on the order of about 16 to 1,000,000 or more diversomers can be synthesized and screened for a particular activity or property.

In an exemplary embodiment, a library of substituted diversomers can be synthesized using the subject reactions adapted to the techniques described in the Still et al. PCT publication WO 94/08051, e.g., being linked to a polymer bead by a hydrolyzable or photolyzable group, e.g., located at one of the positions of substrate. According to the Still et al. technique, the library is synthesized on a set of beads, each bead including a set of tags identifying the particular diversomer on that bead. In one embodiment, which is particularly suitable for discovering enzyme inhibitors, the beads can be dispersed on the surface of a permeable membrane, and the diversomers released from the beads by lysis of the bead linker. The diversomer from each bead will diffuse across the membrane to an assay zone, where it will interact with an enzyme assay. Detailed descriptions of a number of combinatorial methodologies are provided below.

A) Direct Characterization

A growing trend in the field of combinatorial chemistry is to exploit the sensitivity of techniques such as mass spectrometry (MS), e.g., which can be used to characterize sub-femtomolar amounts of a compound, and to directly determine the chemical constitution of a compound selected from a combinatorial library. For instance, where the library is provided on an insoluble support matrix, discrete populations of compounds can be first released from the support and characterized by MS. In other embodiments, as part of the MS sample preparation technique, such MS techniques as MALDI can be used to release a compound from the matrix, particularly where a labile bond is used originally to tether the compound to the matrix. For instance, a bead selected from a library can be irradiated in a MALDI step in order to release the diversomer from the matrix, and ionize the diversomer for MS analysis.

B) Multipin Synthesis

The libraries of the subject method can take the multipin library format. Briefly, Geysen and co-workers (Geysen et al. (1984) PNAS 81:3998-4002) introduced a method for generating compound libraries by a parallel synthesis on polyacrylic acid-grated polyethylene pins arrayed in the microtitre plate format. The Geysen technique can be used to synthesize and screen thousands of compounds per week using the multipin method, and the tethered compounds may be reused in many assays. Appropriate linker moieties can also been appended to the pins so that the compounds may be cleaved from the supports after synthesis for assessment of purity and further evaluation (cf., Bray et al. (1990) Tetrahedron Lett 31:5811-5814; Valerio et al. (1991) Anal Biochem 197:168-177; Bray et al. (1991) Tetrahedron Lett 32:6163-6166).

C) Divide-Couple-Recombine

In yet another embodiment, a variegated library of compounds can be provided on a set of beads utilizing the strategy of divide-couple-recombine (see, e.g., Houghten (1985) PNAS 82:5131-5135; and U.S. Pat. Nos. 4,631,211; 5,440,016; 5,480,971). Briefly, as the name implies, at each synthesis step where degeneracy is introduced into the library, the beads are divided into separate groups equal to the number of different substituents to be added at a particular position in the library, the different substituents coupled in separate reactions, and the beads recombined into one pool for the next iteration.

In one embodiment, the divide-couple-recombine strategy can be carried out using an analogous approach to the so-called “tea bag” method first developed by Houghten, where compound synthesis occurs on resin sealed inside porous polypropylene bags (Houghten et al. (1986) PNAS 82:5131-5135). Substituents are coupled to the compound-bearing resins by placing the bags in appropriate reaction solutions, while all common steps such as resin washing and deprotection are performed simultaneously in one reaction vessel. At the end of the synthesis, each bag contains a single compound.

D) Combinatorial Libraries by Light-Directed, Spatially Addressable Parallel Chemical Synthesis

A scheme of combinatorial synthesis in which the identity of a compound is given by its locations on a synthesis substrate is termed a spatially-addressable synthesis. In one embodiment, the combinatorial process is carried out by controlling the addition of a chemical reagent to specific locations on a solid support (Dower et al. (1991) Annu Rep Med Chem 26:271-280; Fodor, S. P. A. (1991) Science 251:767; Pirrung et al. (1992) U.S. Pat. No. 5,143,854; Jacobs et al. (1994) Trends Biotechnol 12:19-26). The spatial resolution of photolithography affords miniaturization. This technique can be carried out through the use protection/deprotection reactions with photolabile protecting groups.

The key points of this technology are illustrated in Gallop et al. (1994) J. Med Chem 37:1233-1251. A synthesis substrate is prepared for coupling through the covalent attachment of photolabile nitroveratryloxycarbonyl (NVOC) protected amino linkers or other photolabile linkers. Light is used to selectively activate a specified region of the synthesis support for coupling. Removal of the photolabile protecting groups by light (deprotection) results in activation of selected areas. After activation, the first of a set of amino acid analogs, each bearing a photolabile protecting group on the amino terminus, is exposed to the entire surface. Coupling only occurs in regions that were addressed by light in the preceding step. The reaction is stopped, the plates washed, and the substrate is again illuminated through a second mask, activating a different region for reaction with a second protected building block. The pattern of masks and the sequence of reactants define the products and their locations. Since this process utilizes photolithography techniques, the number of compounds that can be synthesized is limited only by the number of synthesis sites that can be addressed with appropriate resolution. The position of each compound is precisely known; hence, its interactions with other molecules can be directly assessed.

In a light-directed chemical synthesis, the products depend on the pattern of illumination and on the order of addition of reactants. By varying the lithographic patterns, many different sets of test compounds can be synthesized simultaneously; this characteristic leads to the generation of many different masking strategies.

E) Encoded Combinatorial Libraries

In yet another embodiment, the subject method utilizes a compound library provided with an encoded tagging system. A recent improvement in the identification of active compounds from combinatorial libraries employs chemical indexing systems using tags that uniquely encode the reaction steps a given bead has undergone and, by inference, the structure it carries. Conceptually, this approach mimics phage display libraries, where activity derives from expressed peptides, but the structures of the active peptides are deduced from the corresponding genomic DNA sequence. The first encoding of synthetic combinatorial libraries employed DNA as the code. A variety of other forms of encoding have been reported, including encoding with sequenceable bio-oligomers (e.g., oligonucleotides and peptides), and binary encoding with additional non-sequenceable tags.

1) Tagging with sequenceable bio-oligomers

The principle of using oligonucleotides to encode combinatorial synthetic libraries was described in 1992 (Brenner et al. (1992) PNAS 89:5381-5383), and an example of such a library appeared the following year (Needles et al. (1993) PNAS 90:10700-10704). A combinatorial library of nominally 77 (=823,543) peptides composed of all combinations of Arg, Gln, Phe, Lys, Val, D-Val and Thr (three-letter amino acid code), each of which was encoded by a specific dinucleotide (TA, TC, CT, AT, TT, CA and AC, respectively), was prepared by a series of alternating rounds of peptide and oligonucleotide synthesis on solid support. In this work, the amine linking functionality on the bead was specifically differentiated toward peptide or oligonucleotide synthesis by simultaneously preincubating the beads with reagents that generate protected OH groups for oligonucleotide synthesis and protected NH₂ groups for peptide synthesis (here, in a ratio of 1:20). When complete, the tags each consisted of 69-mers, 14 units of which carried the code. The bead-bound library was incubated with a fluorescently labeled antibody, and beads containing bound antibody that fluoresced strongly were harvested by fluorescence-activated cell sorting (FACS). The DNA tags were amplified by PCR and sequenced, and the predicted peptides were synthesized. Following such techniques, compound libraries can be derived for use in the subject method, where the oligonucleotide sequence of the tag identifies the sequential combinatorial reactions that a particular bead underwent, and therefore provides the identity of the compound on the bead.

The use of oligonucleotide tags permits exquisitely sensitive tag analysis. Even so, the method requires careful choice of orthogonal sets of protecting groups required for alternating co-synthesis of the tag and the library member. Furthermore, the chemical lability of the tag, particularly the phosphate and sugar anomeric linkages, may limit the choice of reagents and conditions that can be employed for the synthesis of non-oligomeric libraries. In preferred embodiments, the libraries employ linkers permitting selective detachment of the test compound library member for assay.

Peptides have also been employed as tagging molecules for combinatorial libraries. Two exemplary approaches are described in the art, both of which employ branched linkers to solid phase upon which coding and ligand strands are alternately elaborated. In the first approach (Kerr JM et al. (1993) J Am Chem Soc 115:2529-2531), orthogonality in synthesis is achieved by employing acid-labile protection for the coding strand and base-labile protection for the compound strand.

In an alternative approach (Nikolaiev et al. (1993) Pept Res 6:161-170), branched linkers are employed so that the coding unit and the test compound can both be attached to the same functional group on the resin. In one embodiment, a cleavable linker can be placed between the branch point and the bead so that cleavage releases a molecule containing both code and the compound (Ptek et al. (1991) Tetrahedron Lett 32:3891-3894). In another embodiment, the cleavable linker can be placed so that the test compound can be selectively separated from the bead, leaving the code behind. This last construct is particularly valuable because it permits screening of the test compound without potential interference of the coding groups. Examples in the art of independent cleavage and sequencing of peptide library members and their corresponding tags has confirmed that the tags can accurately predict the peptide structure.

2) Non-sequenceable Tagging: Binary Encoding

An alternative form of encoding the test compound library employs a set of non-sequencable electrophoric tagging molecules that are used as a binary code (Ohlmeyer et al. (1993) PNAS 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 2⁴⁰ (e.g., upwards of 10¹²) different molecules. In the original report (Ohlmeyer et al., supra) the tags were bound to about 1% of the available amine groups of a peptide library via a photocleavable o-nitrobenzyl linker. This approach is convenient when preparing combinatorial libraries of peptide-like or other amine-containing molecules. A more versatile system has, however, been developed that permits encoding of essentially any combinatorial library. Here, the compound would be attached to the solid support via the photocleavable linker and the tag is attached through a catechol ether linker via carbene insertion into the bead matrix (Nestler et al. (1994) J Org Chem 59:4723-4724). This orthogonal attachment strategy permits the selective detachment of library members for assay in solution and subsequent decoding by ECGC after oxidative detachment of the tag sets.

Although several amide-linked libraries in the art employ binary encoding with the electrophoric tags attached to amine groups, attaching these tags directly to the bead matrix provides far greater versatility in the structures that can be prepared in encoded combinatorial libraries. Attached in this way, the tags and their linker are nearly as unreactive as the bead matrix itself. Two binary-encoded combinatorial libraries have been reported where the electrophoric tags are attached directly to the solid phase (Ohlmeyer et al. (1995) PNAS 92:6027-6031) and provide guidance for generating the subject compound library. Both libraries were constructed using an orthogonal attachment strategy in which the library member was linked to the solid support by a photolabile linker and the tags were attached through a linker cleavable only by vigorous oxidation. Because the library members can be repetitively partially photoeluted from the solid support, library members can be utilized in multiple assays. Successive photoelution also permits a very high throughput iterative screening strategy: first, multiple beads are placed in 96-well microtiter plates; second, compounds are partially detached and transferred to assay plates; third, a metal binding assay identifies the active wells; fourth, the corresponding beads are rearrayed singly into new microtiter plates; fifth, single active compounds are identified; and sixth, the structures are decoded.

### Exemplification

The invention now being generally described, it will be more readily understood by reference to the following examples which are included merely for purposes of illustration of certain aspects and embodiments of the present invention, and are not intended to limit the invention.

### EXAMPLE 1

General Methods

All chemicals used were reagent grade and used as supplied except where noted. Dichloromethane (CH₂Cl₂) and tetrahydrofuran (THF) used for washing cycles were purchased from Mallinckrodt (HPLC Grade) and used without further purification. CH₂Cl₂ and THF used for reagent preparation were purchased from J. T. Baker (Cycletainer™) and passed through a neutral alumina column prior to use. Pyridine was refluxed over calcium hydride and distilled prior to use. Trimethylsilyl trifluoromethanesulfonate (TMSOTf) was purchased from Acros Chemicals. Sodium methoxide (25% w/v in MeOH), glacial acetic acid (AcOH) and hydrazine acetate (98%) were purchased from Aldrich Chemicals. Fluorous silica (0.77 mmol/g, 230-400 mesh) and isocyanate-3 scavenging resin (1.4 mmol/g, 230-400 mesh) was purchased from Silicycle Inc. Analytical thin-layer chromatography was performed on E. Merck silica gel 60 F₂₅₄ plates (0.25 mm). Compounds were visualized by dipping the plates in a cerium sulfate-ammonium molybdate solution followed by heating. Liquid column chromatography was performed using forced flow of the indicated solvent on Silicycle 230-400 mesh (60 Å pore diameter) silica gel. ¹H NMR spectra were obtained on a Varian VXR-500 (500 MHz) or a Bruker-400 (400 MHz) spectrometer and are reported in parts per million (δ) relative to CHCl₃ (7.27 ppm). Coupling constants (J) are reported in Hertz. ¹³C NMR spectra were obtained on a Varian VXR-500 (125 MHz) or a Bruker-400 (100 MHz) spectrometer and are reported in δ relative to CDC1₃ (77.23 ppm) as an internal reference.

### EXAMPLE 2

General Procedure A: Installation of the A-Tag Ester (Solution Phase)

Alcohol (1.0 equiv.) was dissolved in CH₂Cl₂ (10 mL/mmol) and pyridine (1.0 equiv.). 4-Dimethylaminopyridine (DMAP) (0.1 equiv.) and 2-azido-2-methylpropionic acid (A-Tag) anhydride 2 (1.5 equiv.) were added. The resulting mixture was stirred at ambient temperature for 1 h, after which time the reaction mixture was diluted with CH₂Cl₂, washed with 1% aqueous HCl and water and the organic layer was dried over Na₂SO₄. After filtration and removal of solvents, the product was purified by passing through a plug of silica (eluent: 3:1 hexanes:EtOAc).

### EXAMPLE 3

General Procedure B: Incorporation of the A-Tag Cycle (Automated Solid-Phase)

The resin (25 μmol) was swelled in CH₂Cl₂ (3 mL) and the A-Tag anhydride 2 (30.0 mg, 5 equiv., loaded into cartridges) was dissolved in CH₂Cl₂ (2 mL) and added to the reaction vessel. After vortexing for 5 s, 1 mL of a 0.01 M solution of DMAP in pyridine was added. Mixing of the suspension was performed (10 s vortex, 50 s rest) for 15 min.

### EXAMPLE 4

General Procedure C: Oligosaccharide cleavage from the polymer support and purification (A-Tag Method)

The glycosylated resin (25 μmol) was dried in vacuo over phosphorous pentoxide for 12 h and transferred to a solid-phase round bottom flask. The resin was swelled in THF (3 mL) and tributyl phosphine (112 μL, 18 equiv.) and water (13 μL, 30 equiv.) were added and the reaction mixture was shaken for 30 min. The resin was washed with THF (8×5 mL) and CH₂Cl₂ (8×5 mL) and dried in vacuo over phosphorous pentoxide for 2 h. The resin was transferred to a 10 mL round bottom flask, purged with ethylene and Grubbs' catalyst (bis(tricyclohexylphosphine)benzylidine ruthenium (IV) dichloride, 4.1 mg, 20 mol %) was added. The reaction mixture was diluted with CH₂Cl₂ (3 mL) and stirred under 1 atm ethylene for 36 h. Triethylamine (100 μL, 160 equiv.) and tris hydroxymethylphosphine (50 mg, 80 equiv.) were added and the resulting solution was stirred at room temperature for 1 h. The pale yellow reaction mixture was diluted with CH₂Cl₂ (5 mL) and washed with water (3×5 mL). The aqueous phase was extracted with CH₂Cl₂ (3×5 mL) and the combined organics were dried over Na₂SO₄, filtered and concentrated. The crude mixture was dissolved in CH₂Cl₂ (1 mL) and Silicycle Inc. isocyanate-3 resin (50.0 mg, 1.4 mmol/g loading, 3.0 equiv.) was added. The reaction mixture was stirred for 3 h, filtered and the solvents were removed in vacuo. The product was then either isolated by chromatography or analyzed by HPLC and compared to a pure trisaccharide standard.

### EXAMPLE 5

General Procedure D: Installation of the F-Tag Cap (Solution Phase)

Alcohol (1.0 equiv.) was dissolved in CH₂Cl₂ (10 mL/mmol) and 2,6-lutidine (6.0 equiv.) was added. The solution was stirred for 5 min, then a 0.1 M solution of F-Tag triflate 11 (4.0 equiv.) was added dropwise. After 10 min the clear solution was diluted with CH₂Cl₂ (20 mL), washed with sat. NaHCO₃ (2×25 mL) and brine (25 mL). Following drying (Na₂SO₄), filtration and concentration the crude material was purified by silica gel column chromotography (10% EtOAc/hexanes).

### EXAMPLE 6

General Procedure E: Incorporation of the F-Tag Cap Cycle (Automated Solid-Phase)

The resin (50 μmol) was swelled in a 0.1 M solution of 2,6-lutidine in CH₂Cl₂ (4 mL, 8.0 equiv.). After vortexing for 5 s, a 0.1 M solution of the F-Tag triflate 11 in CH₂Cl₂ (2.5 mL, 5.0 equiv., loaded into cartidges) was delivered to the reaction vessel. Mixing of the suspension was performed (10 s vortex, 50 s rest) for 15 min.

### EXAMPLE 7

General Procedure F: Oligosaccharide Cleavage From the Polymer Support and Purification (F-Tag Method)

The glycosylated resin (50 μmol) was dried in vacuo over phosphorous pentoxide for 12 h and transferred to a round bottom flask. After purging with ethylene, Grubbs' catalyst (bis(tricyclohexylphosphine)benzylidine ruthenium (IV) dichloride, 8.2 mg, 20 mol %) was added. The reaction mixture was diluted with CH₂Cl₂ (1 mL) and stirred under 1 atm ethylene for 36 h. Triethylamine (100 μL, 160 equiv.) and tris hydroxymethylphosphine (50 mg, 80 equiv.) were added and the resulting solution was stirred at room temperature for 1 h. The pale yellow reaction mixture was diluted with CH₂Cl₂ (5 mL) and washed with water (3×5 mL). The aqueous phase was extracted with CH₂Cl₂ (3×5 mL) and the combined organics were dried over Na₂SO₄, filtered and concentrated. The crude mixture was dissolved in CH₂Cl₂/MeOH (1 mL, 1:1) and added to a column of tridecafluoro (Si—(CH₂)₂—(CF₂)₅—CF₃)₃) functionalized silica gel (Silicycle) equilibrated in 80% MeOH/20% H₂O. One column length of 80% MeOH/20% H₂O was eluted, and the solvent was changed to 100% MeOH. Non-fluorinated material typically eluted in fractions 1-4, while fluorinated material remained on the column until the gradient was increased to 100% MeOH. The desired non-fluorinated fractions were concentrated and analyzed by HPLC. Recycling of the fluorous silica gel was possible after washing with 3 column lengths MeOH, 4 column lengths CH₂Cl₂ and drying with N₂.

### EXAMPLE 8

Synthesis of 2-azido-2-methylpropionic Acid Anhydride 2

2-Azido-2-methylpropionic acid was synthesized using known procedures from the commercially available 2-bromo-2-methylpropionic acid. Tornoe, CW, et al., J. Peptide Sci. 2000, 6(7), 314. 2-Azido-2-methylpropionic acid (4.8 g, 37.3 mmol) was dissolved in dry diethyl ether (50 mL) and dicyclohexylcarbodiimide (DCC) (3.8 g, 18.7 mmol) was added. The reaction mixture was stirred overnight, filtered through a pad of celite and washed with saturated NaHCO₃. The organic layer was dried over Na₂SO₄, filtered and the solvents were removed in vacuo to afford 2 (5.93 g, 24.7 mmol, 66%) with no further purification. ¹H-NMR (400 MHz) δ 1.58 (s, 6H); ¹³C-NMR (100 MHz) δ 167.7, 63.8, 24.1.

### EXAMPLE 9

Synthesis of 1,1,1,2,2,3,3,4,4,5,5,6,6,7,7,8,8-heptadecafluorodecyldiisopropylsilyl triflate 11

Triflate 11 was prepared according to the following procedure obtained from Fluorous Technologies Inc., 970 William Pitt Way, Pittsburgh, Pa. 15238; www.fluorous.com. 1,1,1,2,2,3,3,4,4,5,5,6,6,7,7,8,8-Heptadecafluorodecyldiisopropylsilane

To a dry round bottom flask charged with Et₂O (16 mL) was added a 1.7 M solution of tert-butyllithium in pentane (6.15 mL, 10.5 mmol). The yellow solution was cooled to −78° C., stirred for 5 min, and a solution of 1,1,1,2,2,3,3,4,4,5,5,6,6,7,7,8,8-heptadecafluoro-10-iodo-decane (2.0 g, 3.48 mmol) in Et₂O (20 mL) was added dropwise by cannula. The resulting solution was stirred at −78° C. for 1 h, after which diisopropylchlorosilane (505 μL, 2.96 mmol) was added. The mixture was warmed to room temperature over 2 h, quenched with saturated aqueous NH₄Cl and washed with CH₂Cl₂ (3×50 mL). The combined organics were washed with brine (1×50 mL), dried (Na₂SO₄), filtered and concentrated. The crude oil was purified by vacuum distillation (b.p. 78° C. at 0.04 mm) to give the silane as a clear oil (1.4 g, 80%). ¹H NMR (500 MHz) δ 3.49 (br s, 1H), 2.21-2.03 (m, 2H), 1.06 (s, 12H), 0.89-0.82 (m, 2H); ¹³C-NMR (125 MHz) δ 27.2 (t, J=24.2 Hz), 18.8, 18.4, 10.4, −2.16.

1,1,1,2,2,3,3,4,4,5,5,6,6,7,7,8,8-Heptadecafluorodecyldiisopropylsilyl triflate 11

The above silane (2.03 g, 3.61 mmol) was dissolved in CH₂Cl₂ (36 mL) and dry trifluoromethanesulfonic acid (479 μL, 5.41 mmol) was added. The resulting pale yellow solution was stirred at room temperature for 3 h, then transferred and stored in dry vials. Triflate 11 was stable for up to 2 months when stored under N₂ in a dessicator.

### EXAMPLE 10

Automated Synthesis of β-glucoside 14 Using the A-Tag Method

Octenediol functionalized resin 12 (25 μmol, 90 mg, 0.30 mmol/g loading) was loaded into a reaction vessel equipped with a cooling jacket and inserted into a modified ABI-433A peptide synthesizer. The resin was glycosylated using donor 13 (5 equiv., 0.125 mmol, 90 mg loaded into cartridges) delivered in CH₂Cl₂ (3 mL) and TMSOTf (5 equiv., 1 mL, 0.125 M TMSOTf in CH₂Cl₂) at −15° C. Mixing of the suspension was performed (10 s vortex, 50 s rest) for 15 min. The resin was then washed with CH₂Cl₂ (6×4 mL each), warmed to 15° C., and the unglycosylated sites were capped using General Procedure B. Deprotection of the levulinoyl ester was carried out by treating the glycosylated resin with hydrazine acetate (20 equiv., 4 mL, 0.25 M N₂H₄-HOAc in pyridine:acetic acid 3:2) for 15 min. The resin was subjected to the deprotection conditions a second time for 15 min followed by the washing cycle. The deprotected polymer-bound C6—OH β-monosaccharide was then elongated by reiteration of the above glycosylation/capping/deprotection protocol using donor 13. The final trisaccharide was not deprotected, thereby simplifying the analysis of the products. The product was liberated nd purified by General Procedure C.

A-TAG/PHOSPHATE/LEVULINATE CYCLE

| | | | Time |
|---|---|---|---|
| Step | Function | REAGENT | (Min) |
| 1 | Glycosylation | 5 equiv. Donor and 5 equiv. | 20 |
| | | TMSOTf | |
| 2 | Wash | Dichloromethane | 9 |
| 3 | Capping | 5 equiv. 2, cat DMAP | 18 |
| 4 | Wash | Tetrahydrofuran | 9 |
| 5 | Wash | 0.2 M AcOH/MeOH/THF | 9 |
| 6 | Deprotection | 2 × 20 equiv. Hydrazine (NH₂NH₂) | 40 |
| 7 | Wash | Tetrahydrofuran | 9 |
| 8 | Wash | 0.2 M AcOH/MeOH/THF | 9 |
| 9 | Wash | Tetrahydrofuran | 9 |
| 10 | Wash | Dichloromethane | 9 |

### EXAMPLE 11

Automated Synthesis of β-glucoside 14 using the F-Tag Method

Octenediol functionalized resin 12 (50 μmol, 50 mg, 1.0 mmol/g loading) was loaded into a reaction vessel equipped with a cooling jacket and inserted into a modified ABI-433A peptide synthesizer. The resin was glycosylated using donor 13 (5 equiv., 0.250 mmol, 180 mg loaded into cartridges) delivered in CH₂Cl₂ (3 mL) and TMSOTf (5 equiv., 2 mL, 0.125 M TMSOTf in CH₂C₂) was added to the reaction vessel at −15° C. Mixing of the suspension was performed (10 s vortex, 50 s rest) for 15 min. The resin was then washed with CH₂Cl₂ (6×4 mL each), the reaction vessel warmed to 15° C., and the unglycosylated sites were capped using General Procedure E. Deprotection of the levulinoyl ester was carried out by treating the glycosylated resin with hydrazine acetate (20 equiv., 4 mL, 0.25 M N₂H4-HOAc in pyridine:acetic acid 3:2) for 15 min. The resin was subjected to the deprotection conditions a second time for 15 min followed by the washing cycle. The deprotected polymer-bound C6—OH β-disaccharide was then elongated by reiteration of the above glycosylation/capping/deprotection protocol using donor 13. The final trisaccharide was not deprotected, thereby simplifying the analysis of the products. The product was liberated from the resin and purified by General Procedure F.

F-TAG/PHOSPHATE/LEVULINATE CYCLE

| | | | Time |
|---|---|---|---|
| Step | Function | REAGENT | (Min) |
| 1 | Glycosylation | 5 equiv. Donor and 5 equiv. | 20 |
| | | TMSOTf | |
| 2 | Wash | Dichloromethane | 9 |
| 3 | Capping | 5 equiv. 11, 8 equiv. Lutidine | 18 |
| 4 | Wash | Tetrahydrofuran | 9 |
| 5 | Deprotection | 2 × 20 equiv. NH₂NH₂ | 40 |
| 6 | Wash | 0.2 M AcOH/MeOH/THF | 9 |
| 7 | Wash | Tetrahydrofuran | 9 |
| 8 | Wash | Dichloromethane | 9 |

### EXAMPLE 12

n-Pentenyl 3,4-di-O-benzyl-6-O-levulinoyl-2-O-pivaloyl-β-D-glucopyranosyl-(1→6)-3,4-di-O-benzyl-6-O-levulinoyl-2-O-pivaloyl-β-D-glucopyranosyl-(1→6)-3,4-di-O-benzyl-6-O-levulinoyl-2-O-pivaloyl-β-D-glucopyranoside 14

¹H-NMR (500 MHz) δ7.34-7.18 (m, 30H), 5.84-5.76 (m, 1H), 5.09-4.95 (m, 5H), 10H), 4.62-4.55 (m, 3H), 4.52 (d, J=10.6 Hz, 1H), 4.47 (d, J=7.6 Hz, 1H), 4.35 (d, J=7.9 Hz, 1H), 4.34-4.31 (m, 1H), 4.22 (dd, J=5.2, 11.9 Hz, 1H), 4.03-3.96 (m, 2H), 3.88-3.83 (m, 1H), 3.76-3.55 (m, 10H), 3.46-3.42 (m, 3H), 2.67-2.63 (m, 2H), 2.53-2.50 (m, 2H), 2.14 (s, 3H), 2.13-2.06 (m, 2H), 1.69-1.63 (m 2H), 1.20 (s, 9H), 1.19 (s, 9H), 1.18 (s, 9H); ¹³C-NMR (125 MHz)δ206.6, 177.0, 176.9, 176.8, 172.8, 138.3, 138.2, 138.1, 138.0; 128.6, 128.5, 128.4, 128.3, 128.2, 128.1, 128.0, 127.9, 127.8, 115.3, 101.3, 101.0, 100.9, 83.4, 78.4, 78.2, 76.0, 75.4, 75.2, 73.6, 73.4, 73.3, 69.1, 68.4, 67.5, 63.5, 39.3, 38.2, 30.4, 30.2, 29.2, 28.0; ESI-MS m/z (M+Na⁺): Calcd 1485.7119, obsd 1485.7073.

### EXAMPLE 13

Automated Synthesis of α(1→2)-Trimannoside 16 Using the F-Tag Method

Octenediol functionalized resin 12 (50 μmol, 50 mg, 1.0 mmol/g loading) was loaded into a reaction vessel and inserted into a modified ABI-433A peptide synthesizer. The resin was glycosylated using donor 15 (5 equiv., 0.25 mmol, 160 mg) delivered in CH₂Cl₂ (4 mL) and TMSOTf (0.5 equiv., 1 mL, 0.0125 M TMSOTf in CH₂Cl₂). Mixing of the suspension was performed (10 s vortex, 50 s rest) for 30 min. The resin was then washed with CH₂Cl₂ (6×4 mL each) and the unglycosylated sites were capped using General Procedure E. Deprotection of the acetyl ester was carried out by treating the glycosylated resin with sodium methoxide (8 equiv., 0.5 mL, 0.75 M NaOMe in MeOH) in CH₂Cl₂ (5 mL) for 30 min. The resin was then washed with CH₂Cl₂(1×4 mL) and subjected to the deprotection conditions a second time for 30 min. This deprotected polymer-bound C2—OH α-mannoside was then elongated by reiteration of the ation/capping/deprotection protocol. The final trisaccharide was not deprotected, therby simplifying the analysis of the products. The product was liberated from the resin and purified by General Procedure F. For spectral data see: Andrade, R. B. et al., Org. Lett. 1999, 1,

F-TAG/IMIDATE/ACETATE CYCLE

| | | | Time |
|---|---|---|---|
| Step | Function | REAGENT | (Min) |
| 1 | Glycosylation | 5 equiv. Donor and 0.5 equiv. | 20 |
| | | TMSOTf | |
| 2 | Wash | Dichloromethane | 9 |
| 3 | Capping | 5 equiv. 11, 8 equiv. Lutidine | 18 |
| 4 | Wash | Dichloromethane | 9 |
| 5 | Deprotection | 2 × 10 equiv. NaOMe | 60 |
| 6 | Wash | 0.2 M AcOH/MeOH/THF | 9 |
| 7 | Wash | Tetrahydrofuran | 9 |
| 8 | Wash | Dichloromethane | 9 |

### Example 14

6-O-2′-2′-methylpropionoyl-1,2:3,4-di-O-isopropylidene-α-D-galactopyranoside 3

1,2:3,4-Di-O-isopropylidene-α-D-galactopyranoside (50.0 mg, 0.192 mmol) was subjected to General Procedure A to afford 3 (64.0 mg, 0.172 mmol) in 90% yield. [α]²⁴ _{D}:-29.1° (c 2.75,CH₂Cl₂); IR (thin film) 2989, 2937, 2114, 1741, 1069 cm⁻¹; ¹H-NMR (500 MHz) δ5.53 (d, J=5.2 Hz, 1H), 4.62 (dd, J=2.4, 7.9 Hz, 1H), 4.40 (dd, J=4.0, 11.3 Hz, 1H), 4.32-4.27 (m, 2H), 4.24 (dd, J=1.8, 7.9 Hz, 1H), 4.05 (ddd, J=1.8, 4.0, 8.2 Hz, 1H), 1.50 (app s, 6H), 1.48 (s, 3H), 1.46 (s, 3H), 1.35 (s, 3H), 1.33 (s, 3H); ¹³C-NMR (125 MHz) δ173.0, 110.0, 109.0, 96.5, 71.1, 70.9, 70.6, 66.1, 64.8, 63.3, 26.1, 26.1, 25.1, 24.7, 24.6.

### EXAMPLE 15

n-Pentenyl 2-0-2′-Azido-2′-methylpropionoyl-3,4,6-tri-O-benzyl-α-D-mannopyranoside

n-Pentenyl 3,4,6-tri-O-benzyl-α-D-mannopyranoside (61.6 mg, 0.199 mmol) was subjected to General Procedure A to afford product (66.5 mg, 0.106 mmol) in 89% yield. [α]²⁴ D:-20.4° (c 2.67, CH₂Cl₂); IR (thin film) 2924, 2870, 2112, 1743, 1092 cm; ¹H-NMR (500 MHz) δ7.38-7.21 (m, 15H), 5.84-5.75 (m, H), 5.40 (app t, J=2.1 Hz, 1H), 5.06-4.95 (m, 2H), 4.86 (app s, 1H), 4.83 (d, J=10.7 Hz, 1H), 4.70 (d, J=11.3 Hz, 1H), 4.64 (d, J=11.9 Hz, 1H), 4.56 (d, J=11.6 Hz, 1H), 4.52 (d, J=12.8 Hz, 1H), 4.50 (d, J=11.0 Hz, 1H), 4.02-4.00 (m, 1H), 3.87-3.69 (m, 5H), 3.47-3.40 (m, 1H), 2.14-2.08 (m, 2H), 1.71-1.64 (m, 3H), 1.46-1.44 (m, 6H); ¹³C-NMR (125 MHz) δ172.5, 138.5, 138.3, 138.1, 138.1, 128.5, 128.5, 128.3, 128.2, 127.9, 127.9, 127.8, 127.7, 115.3, 97.5, 78.4, 75.5, 74.5, 73.5, 72.0, 71.5, 70.2, 69.1, 67.5, 63.4, 30.4, 28.7, 24.7, 24.6.

### EXAMPLE 16

1,2-3,4-di-O-isopropylidene-6-O-tert-butyldimethylsilyl-α-D-galactop/wanoside 7

1,2:3,4-Di-O-isopropylidene-α-D-galactopyranoside (187 mg, 0.73 mmol) was dissolved in CH₂Cl₂ (1 mL) and 2,6-lutidine (213 μL, 1.83 mmol) was added. The solution was stirred for 5 min and TBSOTf (250 μL, 1.09 mmol) was added dropwise. After 10 min the clear solution was diluted with CH₂Cl₂ (20 mL), washed with saturated NaHCO₃ (2×25 mL) and brine (25 mL). Following drying (Na₂SO₄), filtration and concentration, the crude product was purified by silica gel column chromatography (10% EtOAc/hexanes) to afford 7 (238 mg, 87%) as a clear oil. Spectra are in accordance with the reported data. Dahlhoff, K. M et al., Synthesis 1986, 561.

### EXAMPLE 17

1,2-3,4-di-O-isopropylidene-6-0-(1,1,1,2,2,3,3,4,4,5,5,6,6,7,7,8,8-heptadecafluorodecyldiisopropyl)silyl-α-D-galactopyranoside 8

1,2-3,4-di-O-isopropylidene (14 mg, 0.055 mmol) was subjected to General Procedure D to afford 114 mg of a mixture of 8 and 1,1,1,2,2,3,3,4,4,5,5,6,6,7,7,8,8-heptadecafluorodecyldiisopropylsilyl alcohol. ¹H NMR (500 MHz) δ 5.51 (d, J=5.2 Hz, 1H), 4.61 (dd, J=2.1, 7.9 Hz, 1H), 4.32-4.30 (m, 1H), 4.28 (d, J=8.2 Hz, 1H), 3.87-3.79 (m, 3H), 2.23-2.06 (m, 7H), 1.53 (s, 3H), 1.42 (s, 3H), 1.33 (s, 3H), 1.32 (s, 3H), 1.05-0.97 (m, 42H), 0.89-0.82 (m, 7H); ¹³C-NMR (125 MHz) δ 109.8, 109.2, 100.1, 97.0, 71.4, 71.3, 69.0, 63.0, 26.7, 26.5, 26.1, 25.9, 25.6, 24.9, 18.2, 18.1, 18.1, 17.9, 17.8, 13.5, 13.1, 13.1, 1.36, 0.41.

### Example 18

n-Pentenyl-3,4,6-tri-O-benzyl-2-O-tert-butyldimethylsilyl-a-D-mannopyranoside 9

n-Pentenyl-3,4,6-tri-O-benzyl-α-D-mannopyranoside (55 mg, 0.11 mmol) was dissolved in CH₂Cl₂ (1 mL) and 2,6-lutidine (26 μL, 0.22 mmol) was added. The solution was stirred for 5 min and TBSOTf (27 μL, 0.12 mmol) was added dropwise. After 10 min the clear solution was diluted with CH₂Cl₂ (20 mL), washed with saturated NaHCO₃ (2×25 mL) and brine (25 mL). Following drying (Na₂SO4), filtration and concentration, the crude product was purified by silica gel column chromatography (10% EtOAc/hexanes) to afford 9 (52 mg, 75%) as a clear oil. [α]²⁴ D:+22.8° (c 4.56, CH₂Cl₂); IR (thin film) 2926, 2855, 1497, 1361, 1133 cm⁻¹; ¹H NMR (500 MHz) δ7.41-7.18 (m, 15H), 5.87-5.79 (m, 1H), 5.07-4.98 (m, 4H), 4.84 (d, J=10.7 Hz, 1H), 4.77-4.68 (m, 4H), 4.56 (d, J=11.9 Hz, 1H), 4.53 (d, J=11.0 Hz, 1H), 4.07 (t, J=2.1 Hz, 1H), 3.99-3.95 (m, 1H), 3.85-3.83 (m, 1H), 3.80-3.77 (m, 4H), 3.46-3.42 (m, 1H), 2.16-2.11 (m, 2H), 1.72-1.65 (m, 2H), 0.93 (s, 9H), 0.11 (s, 3H), 0.08 (s, 3H); ¹³C-NMR (125 MHz) δ 138.4, 138.3, 138.1, 137.8, 128.0, 127.9, 127.9, 127.5, 127.3, 127.2, 127.2, 127.1, 127.0, 127.0, 114.6, 100.3, 79.9, 74.7, 74.3, 72.8, 71.9, 69.5, 69.0, 66.5, 30.1, 28.4, 25.5, 17.9, −4.81, −5.10; ESI MS m/z (M+Na⁺) calcd 655.3425, found 655.3449.

### EXAMPLE 19

n-Pentenyl-3,4,6-tri-O-benzyl-2-O-(,1,1,2,2,3,3,4,4,5,5,6,6,7,7,8,8-heptadecafluorodecyldiisopropyl)silyl-α-D-mannopyvranoside 10

n-Pentenyl-3,4,6-tri-O-benzyl-cc-D-mannopyranoside (54 mg, 0.10 mmol) was subjected to General Procedure D to afford 158 mg of a mixture of 10 and 1,1,1,2,2,3,3,4,4,5,5,6,6,7,7,8,8-heptadecafluorodecyldiisopropylsilyl alcohol. ¹H NMR (500 MHz) δ7.39-7.19 (m, 9H), 5.86-5.79 (m, 1H), 5.05 (dd, J=1.53, 17.1 Hz, 1H), 4.98 (dd, J=1.2, 10.1 Hz, 1H), 4.85 (d, J=10.7 Hz, 1H), 4.79-4.72 (m, 2H), 4.67 (d, J=11.9 Hz, 1H), 4.58 (d, J=12.2 Hz, 1H), 4.53 (d, J=11.0 Hz, 1H), 4.12-4.11 (m, 1H), 3.98-3.93 (m, 1H), 3.87 (dd, J=2.4, 9.5 Hz, 1H), 3.81-3.72 (m, 2H), 3.47-3.42 (m, 1H), 2.30-2.11 (m, 4H), 1.74-1.67 (m, 2H), 1.06 (s, 17H), 0.95-0.85 (m, 3H); ¹³C-NMR (125 MHz) δ139.3, 139.2, 139.0, 139.0, 138.9, 138.7, 138.7, 129.0, 128.9, 128.8, 128.7, 128.6, 128.5, 128.3, 128.3, 128.2, 128.1, 121-108 (m, CF₂, CF₃), 101.1, 101.1, 83.3, 80.6, 76.5, 75.8, 75.6, 74.0, 73.9, 73.8, 73.0, 72.6, 71.8, 71.6, 69.9, 67.6, 31.0, 30.4, 29.3, 26.2, 26.0, 26.0 (t, J=21.9 Hz), 18.3, 18.3, 18.2, 18.2, 17.8, 17.8, 13.8, 13.7, 13.6, 13.5, 13.3, 1.5, 1.4.

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of facilitating the purification of compounds synthesized on a solid support, comprising the step of: treating a compound covalently tethered to a solid support, wherein said compound comprises a nucleophilic functional group, with a capping-tagging reagent selected from the group consisting of activated azido-carboxylic acids, azido-carboxylic acid anhydrides, fluoroalkylsilyl sulfonates, and fluoroalkylsilyl halides, thereby forming a second compound tethered to a solid support.

2. The method of claim 1, wherein the compound covalently tethered to a solid support is a biopolymer.

3. The method of claim 1, wherein the compound covalently tethered to a solid support is an oligosaccharide.

4. The method of claim 1, wherein the compound covalently tethered to a solid support is an oligonucleotide.

5. The method of claim 1, wherein the nucleophilic functional group is an alcohol.

6. The method of claim 1, wherein the nucleophilic functional group is an amine.

7. The method of claim 1, wherein the capping-tagging reagent is an activated 2-azido-carboxylic acid or 2-azido-carboxylic acid anhydride.

8. The method of claim 1, wherein the capping-tagging reagent is a 2-azido-carboxylic acid anhydride.

9. The method of claim 1, wherein the capping-tagging reagent is 2-azido-isobutyric acid anhydride.

10. The method of claim 1, wherein the capping-tagging reagent is a fluoroalkylsilyl triflate.

11. The method of claim 1, wherein the capping-tagging reagent is a fluoroalkyldiisopropylsilyl triflate.

12. The method of claim 1, wherein the capping-tagging reagent is heptadecafluorodecyldiisopropylsilyl triflate.

13. The method of claim 1, wherein the compound covalently tethered to a solid support is an oligosaccharide; and the nucleophilic functional group is an alcohol.

14. The method of claim 1, wherein the compound covalently tethered to a solid support is an oligonucleotide; and the nucleophilic functional group is an alcohol.

15. The method of claim 1, wherein the compound covalently tethered to a solid support is an oligosaccharide; the nucleophilic functional group is an alcohol; and the capping-tagging reagent is 2-azido-isobutyric acid anhydride.

16. The method of claim 1, wherein the compound covalently tethered to a solid support is an oligonucleotide; the nucleophilic functional group is an alcohol; and the capping-tagging reagent is 2-azido-isobutyric acid anhydride.

17. The method of claim 1, wherein the compound covalently tethered to a solid support is an oligosaccharide; the nucleophilic functional group is an alcohol; and the capping-tagging reagent is heptadecafluorodecyldiisopropylsilyl triflate.

18. The method of claim 1, wherein the compound covalently tethered to a solid support is an oligonucleotide; the nucleophilic functional group is an alcohol; and the capping-tagging reagent is heptadecafluorodecyldiisopropylsilyl triflate.

19. The method of any of claims 1-18, further comprising the steps of: cleaving said tether of said second compound covalently tethered to a solid support to produce a compound in solution; and purifying said compound in solution using affinity chromatography or a scavenging resin.

20. The method of claim 19, wherein said compound in solution is purified using affinity chromatography with a fluorous silica gel as a stationary phase.

21. The method of claim 19, wherein said compound in solution is purified using a scavenging resin comprising an isocyanate, isothiocyanate, alkyl halide, aryl halide, aryl boronic acid, alkyl sulfonate, aldehyde, carboxylic acid halide, carboxylic acid anhydride, or sulfonyl halide.

22. The method of claim 19, wherein said compound in solution is purified using a scavenging resin comprising an isocyanate or isothiocyanate.
